(19) United States
(12) Patent Application Publication
(10) Pub. No.: US 2010/0002233 A1
(43) Pub. Date: Jan. 7, 2010

(54) IMAGING OF TURBID MEDIUM

(75) Inventors: Maarten Marinus Johannes Wilhelm Van Herpen, Eindhoven (NL); Martinus Bernardus Van Der Mark, Eindhoven (NL); Michael Cornelis Van Beek, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/447,669

(22) PCT Filed: Oct. 24, 2007
(86) PCT No.: PCT/IB07/54323

§ 371 Date: Apr. 29, 2009

(30) Foreign Application Priority Data
Oct. 30, 2006 (EP) 06123182.5

(51) Int. Cl. A61B 5/00; G01N 21/00

(52) U.S. Cl. 356/432

(57) ABSTRACT

The invention relates to imaging of a turbid medium, for example in connection with optical mammography. A device for imaging a turbid medium (20) is disclosed, the device comprising: a holder (20) arranged for receiving the turbid medium and a matching fluid (21); one or more radiation sources (24) and one or more photodetectors (25). The matching fluid is a vapor with one or more optical properties of the matching fluid substantially matching the corresponding one or more optical properties of the turbid medium. In an embodiment, the matching fluid (21) is a composite vapor comprising at least two components.

### FIELD OF THE INVENTION

The invention relates to a device for imaging a turbid medium, and in particular by imaging the turbid medium by means of optical radiation. Moreover, the invention relates to a method of imaging a turbid medium.

### BACKGROUND OF THE INVENTION

A number of devices for imaging the internal structure of human or animal tissue exists, a type of such devices pertains to optical mammography for in vivo examinations of breast tissue of a human or animal female. In this case, the turbid medium is the breast of the female to be examined.

In known types of optical mammography devices, the breast or part of the breast is put inside a holder including a number of light sources and photodetectors which are distributed across the wall of the holder. The holder moreover contains a matching liquid in which the breast is immersed. The matching liquid provides optical coupling between the part of the breast to be imaged and the light sources and the photodetectors, respectively. Furthermore, the optical parameters of the matching liquid, such as the reduced scattering coefficient μₛ' and the absorption coefficient μₐ, are selected to be approximately equal to those of the part of the breast to be imaged. The matching liquid prevents optical short-circuiting between the light sources and the photodetectors, moreover, the matching liquid also counteracts boundary effects in the reconstructed image; such effects are caused by the difference in optical contrast between the interior of the breast tissue and the remaining space in the holder. In order to measure the intensities, alternately one of the light sources irradiates the part of the breast to be imaged and the photodetectors measure a part of the light transported through the part of the breast to be imaged. These measurements are repeated until the part to be imaged has been irradiated by all light sources present in the holder, and an image of the interior of the part of the breast to be imaged may subsequently be reconstructed from the measured intensity measurements.

U.S. Pat. No. 5,907,406 discloses a device for imaging a turbid medium. The device includes a holder, a light source, a photodetector and a processing unit. The holder is adapted to receive besides the turbid medium also a liquid adaptation medium having substantial identical optical parameters as the optical parameters of the turbid medium. A drawback of this method is that the patient will always need to lie down, because the measurement can only be done with the breast hanging down into the liquid, since otherwise the liquid will leak out. A general problem with absorbing/scattering liquids, is that the scattering/absorbing particles within the liquid will be pulled down by gravity and hence need to be stabilized to prevent settling of the particles on the bottom.

The inventor of the present invention has appreciated that an improved way of imaging a turbid medium, such as in connection with optical mammography, may be of benefit, and has in consequence devised the present invention.

### SUMMARY OF THE INVENTION

The present invention addresses the above needs by providing an improved way of imaging turbid medium, and preferably, the invention alleviates, mitigates or eliminates one or more of the above or other disadvantages singly or in any combination. To this end, the inventors have had the insight that, until now, liquid media have been used as adaptation medium for matching the optical properties of the adaptation medium and the turbid medium.

According to a first aspect of the present invention there is provided, a device for imaging a turbid medium, the device comprising:

a holder arranged for receiving the turbid medium and a matching fluid;

one or more radiation sources for irradiating the turbid medium and the matching fluid;

one or more photodetectors for measuring the intensity of the radiation;

wherein the matching fluid is a vapor with one or more optical properties of the matching fluid substantially matching the corresponding one or more optical properties of the turbid medium.

In an embodiment, the device is a device for performing optical mammography.

In the context of this application, vapor is to be understood in a broad sense, and at least to include gaseous solid particles, liquid particles, aerosols and particulate matter in general suspended in an atmosphere or ambient, such as air.

The invention is particularly, but not exclusively advantageous, for providing a device which solves the short-circuit problem in connection with imaging of turbid medium, which maintains most if not all of the advantages of using a liquid matching fluid, and which moreover allows the patient to sit or stand during the measurement.

In advantageous embodiments the matching fluid is a composite vapor comprising at least two components. By using a composite vapor, an intrinsically dilute medium such as vapor can be provided with sufficient optical density.

To this end, the optical properties of the vapor can be controlled in a number of ways, advantageous embodiments are provided in the dependent claims. It is an advantage that the optical properties of the vapor can be adjusted and controlled in a number of ways, thereby rendering possible a versatile matching fluid.

In an advantageous embodiment the device may further comprise a nebulizer and wherein the vapor is generated in the form of a nebula by the nebulizer.

In an advantageous embodiment the device may further comprise a device for generating sound waves for randomizing the position of the particles in the vapor. It is an advantage to randomize the position of the particles in order to stabilize the optical properties of the matching fluid on the time-scale of a measurement.

In a second aspect, the present invention relates to a method of imaging a turbid medium, the method comprising:

arranging in a holder the turbid medium and a matching fluid;

irradiating the turbid medium and the matching fluid with one or more radiation sources;

measuring the intensity of the radiation by one or more photodetectors;

wherein the matching fluid is selected as a vapor with one or more optical properties of the matching fluid substantially matching the corresponding one or more optical properties of the turbid medium.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 illustrates the short-circuit problem present in optical mammography;

FIG. 2 illustrates an embodiment of a holder of a mammography device;

FIG. 3 shows a graph of the scattering efficiency Q of TiO₂ particles in liquid water as a function of the size parameter x;

FIG. 4 shows a graph of the scattering efficiency Q of water droplets in air as a function of the size parameter x;

FIG. 5 is a schematic illustration of droplets filled with a high concentration of scattering particles;

FIG. 6 illustrates a method of imagining a turbid medium in accordance with the present invention.

### DESCRIPTION OF EMBODIMENTS

One of the challenges for optical mammography is to prevent light from finding a path from the light source to the detector without traveling through the tissue under investigation, i.e. to solve the short-circuit problem.

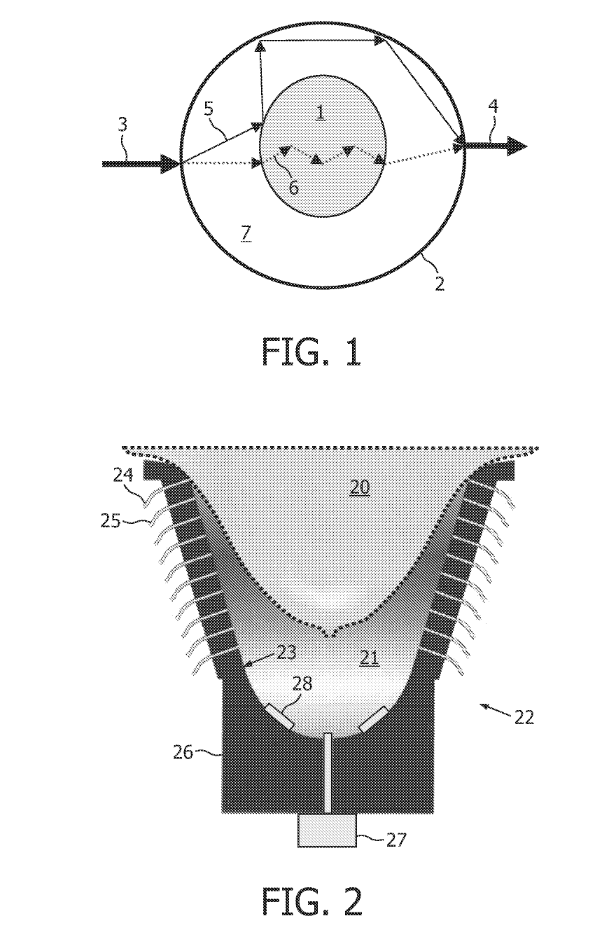

FIG. 1 illustrates the short-circuit problem present in optical mammography. The tissue under investigation, i.e. the turbid medium 1, being a female breast or part of the breast is put inside a holder 2, also often referred to as a cup. The holder also contains the optics, being a light source 3 and detector 4 (or number of light sources and detectors). The solid line 5 shows a path from source 3 to detector 4 traveling around the tissue under investigation. The problem with this is that the small fraction of the light reaching the detector that has traveled through the tissue, as illustrated by the broken line 6, is masked by the comparatively large amount of light which has reached the detector by traveling around the tissue under investigation.

To avoid or at least diminish the short circuit problem the breast is immersed in a fluid 7 provided in the holder. Moreover, by the provision of the fluid one also seeks to achieve the objectives of providing a homogeneous reference medium for calibration, eliminating or diminishing boundary effects due to both container and breast and provides a stable optical contact between optodes and breast. In order to achieve these objectives the optical properties of breast and fluid (scattering, absorption and refractive index) are substantially matched. For example, the match of the attenuation constant K may be within 30%, such as within 20%, such as within 10%, or even better. The match of scattering coefficients, absorption coefficients and refractive indices, may deviate by larger factors, and a match may be within 50%, such as within 30%, such as within 10%, or even better.

FIG. 2 illustrates an embodiment of a holder of a mammography device 22 in accordance with the present invention. A breast 20 is positioned in the holder 26 filled with a vapor 21 to fill up the area between the breast 20 and the cup walls 23. One or more of the optical properties of the matching fluid, i.e. the vapor, is provided such that the one or more optical properties of the matching fluid substantially matching the corresponding one or more optical properties of the turbid medium, i.e. the breast tissue. As a result of the use of a matching fluid having substantially matched optical properties as the turbid medium, an optical short-circuit from the source is prevented or at least suppressed, and the optical properties along the light paths between the light source and the photodetector are rendered similar in all positions. The holder is provided with a set of radiation sources 24 for irradiating the turbid medium and the matching fluid. The radiation sources are typically in the form of fibers attached to the holder so that light can be coupled into the holder. The light can then travel from the sources fibers, through the breast 20 and is then coupled into a series of photodetectors 25 for measuring the intensity of the radiation. The detectors are coupled to the holder by means of fibers attached to the holder. In alternative embodiments, the detectors, such as photodiodes, CCD-chips, etc. may be attached directly on or in the holder.

The holder 22 is part of an optical mammography device, such a device is known e.g. from U.S. Pat. No. 6,480,281 which is hereby incorporated by reference. In order to reconstruct an image of the interior part of the breast to be examined, an iterative method may be applied. Such a method is known e.g. from the patent application WO 99/03394 which is hereby incorporated by reference. The mammography device typically also includes or is connected to a processing unit for deriving an image of the turbid medium from the measured intensities. Moreover, the device may be provided with or connected to a display for displaying the derived image.

Optical properties of opaque or dense media, including opaque fluids and turbid media, may be described in a number of ways. Such media are characterized by at least four parameters (see e.g. H. C. van de Hulst, “Light scattering by small particles”, Dover, N.Y., 1981):

1. The extinction length lₑₓₜ, which is characteristic for loss of intensity in the directly transmitted (unscattered) light: I=I₀exp(-d/lₑₓₜ) due to both absorption and scattering, where I₀ is the incident intensity. For substantially white (non-absorbing) media lₑₓₜ could be replaced with l_{sca}, the scattering mean-free path.

2. The transport mean-free path lₜᵣₐ, which is the effective diffusion length in the bulk of the scattering medium. It is the characteristic length over which the light looses correlation with its original propagation direction.

3. The absorption length l_{abs}, which is indicative of the “whiteness” of the medium.

4. The size or thickness d of the medium

The difference between the scattering mean free path l_{sca} and transport mean free path lₜᵣₐ is a consequence of anisotropic scattering. The following relation holds:

lₜᵣₐ=l_{sca}/(1-

[symbol]

cosθ [symbol]
where θ is the scattering angle. If the particles scatter equal amounts of light in all directions then the mean cosine of the scattering angle is zero and hence lₜᵣₐ=l_{sca}.

The scattering anisotropy is g=<cos(θ)>.

In the above the (statistical) homogeneity of the medium in both space and time is assumed. In space, the medium can indeed have a scattering length scale for scattering, l_{sca}, but also for example, a fractal microstructure associated with a whole range of length scales. In particular, a medium consisting of two scattering length scales is possible, e.g. a cloud of scattering droplets consisting of a scattering suspension of particles. All parameters mentioned relate in some way or another to the optical density of the medium.

A number of parameters and relations are available for statistically homogeneous medium of volume V where: r being the particle radius, n the particle refractive index, n_{med} the refractive index of the medium, λ the wavelength in vacuum, N the number of particles and n₀=N/V the number density of particles; a non-exclusive list of such parameter and relations include:

volume fraction: f=4πr³n₀/3, 0<f<1, typically f<0.7

size parameter: x=2π r n_{med}/λ

geometric cross section: σ_{geo}=π r²

scattering cross section: σ_{sca}

absorption cross section: σ_{abs}

total cross section or extinction cross section: σₑₓₜ=σ_{sca}+σ_{abs}

extinction length: lₑₓₜ=(n₀σₑₓₜ)⁻¹

particle “whiteness” or albedo: a=σ_{sca}/σₑₓₜ

quality factor for scattering: Q_{sca}=σ_{sca}/σ_{geo}

scattering mean free path: l_{sca}=(n₀σ_{sca})⁻¹

scattering coefficient: μₛ=1/l_{sca}

inelastic length: lᵢₙ=al_{sca}/(1-a)=lₑₓₜ/(1-lₑₓₜ/l_{sca})=(lₑₓₜ⁻¹-l_{sca}⁻¹)⁻¹

cross section for radiation pressure: σₚᵣ

quality factor for momentum transfer: Qₚᵣ=σₚᵣ/σ_{geo}

transport mean free path: lₜᵣₐ=(n₀σₚᵣ)⁻¹

reduced scattering coefficient: μₛ'=1/lₜᵣₐ

attenuation length: lₐₜₜ=lₜᵣₐ/√(3(1-a)lₜᵣₐ/(al_{sca}))=√(lₜᵣₐlᵢₙ/3)

absorption coefficient: μₐ=μₛ(1-a)/a

attenuation coefficient: κ=√(3μₐμₛ')=√(3(1-a )/(al_{sca}lₜᵣₐ))=√(3_{μs}'(lₑₓₜ⁻¹-l_{sca}⁻¹))

In general one or more optical properties of the matching fluid may be such that they substantially match the corresponding optical properties of the turbid medium. The radiation source may irradiate the turbid medium at a selected wavelength and for this selected wavelength, the one or more selected optical properties of the matching fluid may substantially be such that they substantially match the corresponding optical properties of the turbid medium. The one or more matching optical properties may be one or more attenuation coefficients, scattering coefficients, absorption coefficients, refractive indices, or other of the above mentioned properties or other optical properties.

The matching fluid may in different embodiments be provided by different types of vapor.

In an embodiment, the vapor is in the form of mist or fog (hereafter only referred to as mist). Mist consists of small liquid droplets giving rise to scattering and absorption. If the mist is dense enough, it is possible to block an optical short-circuit running through the mist. Mist can e.g. be generated from a boiling liquid.

In an embodiment, the vapor is in the form of a cloud of micro-particles. One type of a cloud of micro-particles is smoke, which is composed of small carbon micro-particles.

The requirement for the smoke density can be estimated from air quality tables correlating the concentration of particles in the air with long scale visibility, it may thereby be estimated that the smoke density should be such as 0.24 g/l, such as 0.15 g/l or higher.

In an alternative estimate, one may calculate the required optical density (OD) and compare this density to experimentally obtained OD. The OD is given as:

OD=−¹⁰log(Transmittance pr. meter)

For the female breast, μₛ'≅1 mm⁻¹ (reduced scattering coefficient) and κ=√(3μₛ'μₐ)≅100 m⁻¹ (κ being the attenuation coefficient, and μₐ being the absorption coefficient) the transmittance of the female breast is approximately 1/e in 1 mm, giving an OD of 430. Such OD can be obtained, e.g. from smoke generated from burning certain thermoplastics, such as LATENE 3 H2W-V0 obtainable from LATI Industria Termoplastici (www.lati.com).

In an embodiment, the vapor is in the form of a powder of small particles which are swept through the holder using sound waves.

In an embodiment, a vapor is in the form of a cloud of micro-particles may be generated by means of a ‘nebulizer’, an advantage of a nebulizer is that the resulting vapor feel dry and cold, and may therefore feel more pleasant on the skin. A nebulizer may be applied for generating a cloud of liquid micro-droplets, i.e. a mist. The expelled cloud of micro-particles from the nebulizer is also referred to as a nebula.

For the various embodiments, the amount of scattering and absorption, i.e. the optical properties, can be tuned by adjusting the size (droplet size, particle size), amount and composition of the droplets or micro-particles. It may be important that the effective (statistical) optical properties of the vapor do not change during the measurement. In an embodiment this may be obtained by giving the particulate matter (droplet, particle) of the vapor a sufficiently fast and random movement, so the location of the particulate matter is averaged out. This may be achieved by randomizing the position of the particulates within the timeframe of one measurement to be completed. This may be in the range of 1 ms to 50 ms, such as 25 ms.

In an embodiment, the location of the particulate matter of the vapor is averaged out by the application of high-frequency sound oscillations. Sufficient motion of the particles is obtained by tuning the frequency and the amplitude of the sound waves. Sound with a period of 25 ms corresponds to a frequency of 40 Hz. In order to ensure sufficient randomization a higher frequency sounds may be used, such as 400 Hz or higher. In an embodiment ultra-sound may be used. It is advantageous to use ultra-sound since the patient will not hear the sound. It is however important to ensure that standing wave patterns are not formed in the holder, this may be achieved by chirping the sound frequency, where that the frequency is constantly rapidly changed. In FIG. 2 ultra-sound transducers 28 are schematically illustrated e.g. in the form of piezo transducers, provided on the inside of the holder 22.

As mentioned above, a nebulizer may in an embodiment be utilized for generating the matching fluid, i.e. the vapor inside the holder. A nebulizer is also referred to as an atomizer. Nebulizers are typically used to deliver drugs into the lungs. Different types of nebulizers may be applied, such as compressed air nebulizers, jet nebulizers, and ultrasound nebulizers. In an ultrasound nebulizer vibrations in the MHz range are used to atomize the liquid to micron-size particles (aerosols) which are ejected from a nozzle of the nebulizer. In FIG. 2 a nebulizer 27 is schematically illustrated, the nebulizer being equipped with a nozzle which is inserted into the holder through an opening in the holder. In alternative embodiments, a nebulizer may be included in the holder.

A nebulizer may be driven with pure water to generate a cloud of liquid micro-droplets, however it may be difficult to generate a vapor which is dense enough to obtain high enough extinction.

A denser vapor can be provided by a composite vapor comprising at least two components. The vapor may comprise a first component, also referred to as a first scattering component dissolved in droplets of a second component. To this end, a liquid solution of TiO₂ may be applied so as to generate a cloud of micro-droplets of water with TiO₂ particles dissolved in them, such as TiO₂ nano- or micro-particles. An advantage of using a first scattering component dissolved in a second component, is that the average scattering and absorbing properties of the generated cloud of micro-particles can be tuned to the required values by changing the concentration of the scattering component, e.g. TiO₂ particles inside the droplets as mentioned above. In particular the so-called anisotropy factor or g factor for light scattering can be tuned, and it can be tuned to be much smaller than 1 for the droplets.

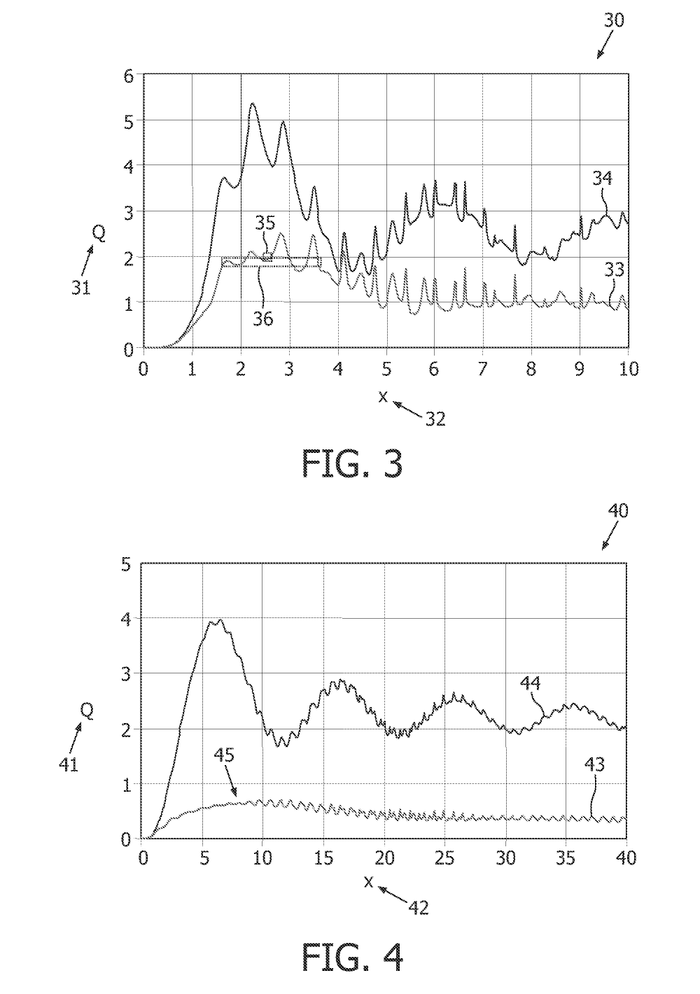

FIG. 3 shows a graph 30 of the scattering efficiency Q 31 of TiO₂ particles in liquid water as a function of the size parameter 32, x=2π r n_{med}/λ. The quality factor for momentum transfer Qₚᵣ is shown as denoted 33, as well as the quality factor for scattering Q_{sca} as denoted by 34.

A liquid scattering medium resembling the female breast may be provided by TiO₂ particles (anatase, n=2.5) of approximately d=2r=250 nm diameter suspended in water (n_{med}=1.327). It is found experimentally that a concentration of p=1.2 g/l gives realistic results at a wavelength of λ=780 nm. Typically the concentration of the TiO₂ particles in the vapor droplets will be higher than in the case of using a pure liquid instead of a gas. Therefore the TiO₂ concentration will be greater than 1.2 g/l (in first approximation by the reciprocal of the liquid volume fraction in the vapor).

Using the TiO₂ specific density of pₛ=4.2 kg/l, a volume fraction of TiO₂ in water is found as f=ρ/ρₛ=4π r³n₀/3=2.86×10⁻⁴, giving a size parameter x=2.67. From FIG. 3, a value for Qₚᵣ=2 is read (as indicated on the graph by reference number 35), and the reduced scattering coefficient is obtained as: μₛ'=lₜᵣₐ⁻¹=n₀ σ_{geo}Qₚᵣ=3ρ Qₚᵣ/(4r ρₛ)=1.72×10³ m⁻¹. The attenuation coefficient is calculated to be κ=√(3 μₐμₛ')=108.7 m⁻¹ and is mainly determined by water absorption with κ=1.44×10⁻⁷, which corresponds to an absorption length, or rather the inelastic length, in pure water of: lᵢₙ=λ/(4πκ)=μₐ⁻¹=0.437 m.

Both the particle and medium refractive index are in fact complex numbers, n-ik, but in case of diffuse scattering, the imaginary parts of both are small compared to the real parts.

Suitable TiO₂-particle size for a TiO₂/water mixture with Qₚᵣ=2.0 has a size parameter of: 1.5<x<3.5 (as indicated on FIG. 3 by reference number 36). This implies particle diameters of 0.28<d<0.65 micron and a volume fraction 0.00014<f<0.00033, if the reduced scattering coefficient is taken to be: μₛ'=1.5 mm⁻¹.

FIG. 4 shows a graph 40 of the scattering efficiency Q41 of water droplets in air as a function of the size parameter 42, x=2π r n_{med}/λ. Both the quality factor for momentum transfer Qₚᵣ is shown as denoted 43, as well as the quality factor for scattering Q_{sca} as denoted by 44.

The best droplet size for water/air mixture (mist) is 5<x<15 with Qₚᵣ=0.6 (as indicated on FIG. 4 by reference number 45). This implies droplet diameters of 1.24<d<3.72 micron and a volume fraction 0.0021<f<0.0062 if we use that μₛ'=3f Qₚᵣ/(4r) and demand that the reduced scattering coefficient is: μₛ'=1.5 mm⁻¹.

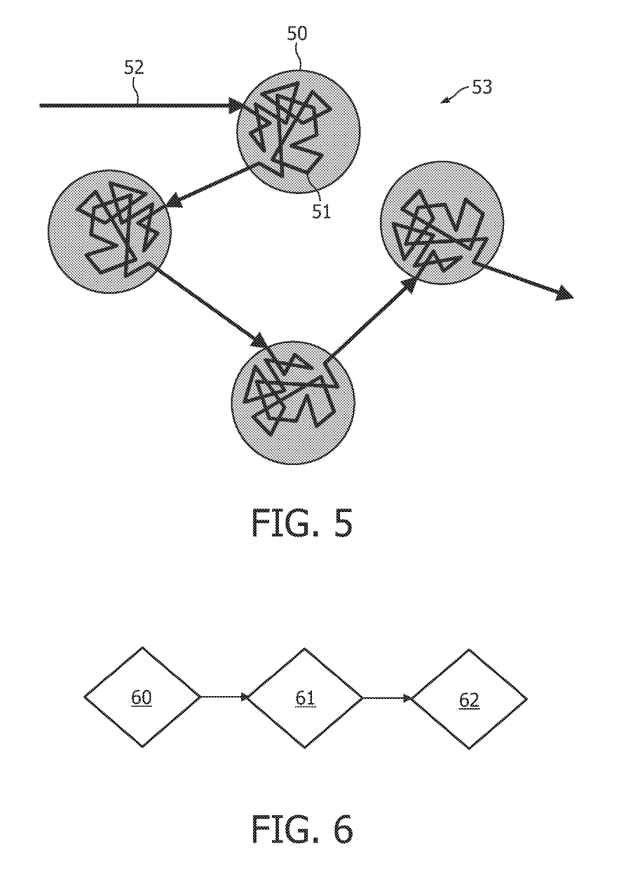

When comparing droplets and (TiO₂) particles due to their size, for the same volume fraction, the droplets typically have a relatively long scattering (transport) length μₛ'. However, when the droplets are filled with a high concentration of TiO₂ particles as illustrated in FIG. 5 there is a high amount of scattering within the droplet, and the light is mainly scattered backwards from the droplet. For droplets alone the scattering is anisotropic in forward direction. For TiO₂ particles suspended in water the scattering is almost isotropic. With TiO₂ particles inside the droplets, the scattering becomes anisotropic in backwards direction and this is an advantage for using droplets with TiO₂ particles.

FIG. 5 is a schematic illustration of a composite vapor 53 consisting of two components. A first scattering component 51 dissolved in droplets 50 of a second component.

The size of the droplets may be large compared to the wavelength of the radiation or light 52. The vapor may in an embodiment be a cloud of micro-droplets 50 of water, filled with a high concentration of TiO₂ particles 51. Such droplets give rise to a two stage scattering mechanism: the light is strongly scattered by the droplets because the droplets contain strong scatters themselves. At high enough concentration of TiO₂ particles the scattering from the droplets will be mainly backwards.

For water droplets that have scattering TiO₂ particles inside, as depicted in FIG. 5, the volume fraction of TiO₂ can be made considerably higher than the corresponding volume fraction of TiO₂ particles dissolved in liquid water. In case the droplet size would be 1 micron, the transport mean free path lₜᵣₐ inside the droplet could be made a fraction of that, such as a quarter of a micron so that the droplet becomes substantially opaque and the quality factor for momentum transfer of the droplet Qₚᵣ rises to a value of the order of 2 and the mean optical path in the droplet will be of the order of 4lₜᵣₐ. When using a nebulizer with TiO₂ particles dissolved in water to generate a composite vapor, the volume fraction for droplets in the nebula may be decreased by a factor of 3 using this approach, assuming that the reduced scattering coefficient remains unaltered.

In general it may be an advantage to select for a component of the composite vapor with a transport mean-free path, lₜᵣₐ, below 3 millimeter or such as below 1 millimeter. Moreover, it may also be advantageous to generate droplets, i.e. the second component, with a size so the droplets in average are larger than the transport mean-free path of the scattering component of the first component. Thus, water droplets may be generated with a larger size than the transport mean-free path of the suspended TiO₂ particles.

Since both the scattering component (dissolved particles) and the droplets of the second component may have light absorbing characteristics, and since both may be tuned within a range of values, it may be an advantage to ensure that contrast of refractive index, i.e. the ratio between the refractive index of the first scattering component and the second component (n/n_{med}), is as large as possible, such as larger than 1.5. The scattering properties of the material as a whole, is determined by the contrast of refractive index.

In general the optical properties of the vapor may be tuned such that the scattering and absorption properties are higher than those of water (droplets).

The attenuation coefficient of the droplet can be adjusted by dissolving an absorbing dye in the droplets. To obtain a value of κ=100 m⁻¹, using just water and dye, the albedo can be calculated using: κ=√(3μₐμₛ')=√(3(1-a)/(al_{sca}lₜᵣₐ))=√(3 μₛ'(lₑₓₜ⁻¹-l_{sca}⁻¹)). From FIG. 4 it can be seen that Q_{sca} varies between 1.7 and 4 over the range 5<x<15.

Consider the two cases x=6 or d=1.49 micron with Q_{sca}=3.9 and f=0.0025 and also x=12 or d=2.98 micron with Q_{sca}=1.8 and f=0.0050, both with Qₚᵣ=0.6, where it is used that μₛ'=3f Qₚᵣ/(4r) and that the reduced scattering coefficient: μₛ'=1.5 mm⁻¹. In this situation κ=μₛ'√(3(a⁻¹-1) Q_{sca}/Qₚᵣ), and hence we find for κ=100 m⁻¹ and x=6 that the albedo a=0.999772 and for x=12 that a=0.995064. The combination of internal absorption in the droplet of both the water and the dye should give rise to the albedo calculated here. The proper values for the complex refractive index can be found by iteratively solving the exact solution for scattering from an absorbing sphere (Mie theory). Given n=1.327, the result for x=6 is κ=1.6×10⁻⁴ and Qₑₓₜ=3.882, Q_{sca}=3.881, Qₚᵣ=0.582, and for x=12 is κ=3.5×10⁻⁵ and Qₑₓₜ=1.660, Q_{sca}=1.651, Qₚᵣ=0.5682. The absorption of water at 780 nm is κ=1.44×10⁻⁷, and hence some absorbing dye must be added. To obtain an absorption of κ=1.6×10⁻⁴, one should look at a dye solution in water with an absorption length (1/e) of l_{abs}=λ/(4π κk)=0.388 mm.

FIG. 4 can only be used to evaluate Q_{sca} in case of weak absorption, but rigorous calculation of the scattering properties of strongly absorbing particles is also possible.

FIG. 6 illustrates a method of imagining a turbid medium in accordance with the present invention, the method may at least comprise the steps of arranging in a holder 60 the turbid medium and a matching fluid; irradiating the turbid medium 61 and the matching fluid with one or more radiation sources; and measuring the intensity of the radiation 62 by one or more photodetectors.

Although the present invention has been described in connection with the specified embodiments, it is not intended to be limited to the specific form set forth herein. Rather, the scope of the present invention is limited only by the accompanying claims. In the claims, the term “comprising” does not exclude the presence of other elements or steps. Additionally, although individual features may be included in different claims, these may possibly be advantageously combined, and the inclusion in different claims does not imply that a combination of features is not feasible and/or advantageous. In addition, singular references do not exclude a plurality. Thus, references to “a”, “an”, “first”, “second” etc. do not preclude a plurality. Furthermore, reference signs in the claims shall not be construed as limiting the scope.

1. A device for imaging a turbid medium (1, 20), the device comprising:
a holder (20) arranged for receiving the turbid medium and a matching fluid (7, 21, 53);
one or more radiation sources (3, 24) for irradiating the turbid medium and the matching fluid;
one or more photodetectors (4, 25) for measuring the intensity of the radiation;
wherein the matching fluid is a vapor with one or more optical properties of the matching fluid substantially matching the corresponding one or more optical properties of the turbid medium.

2. The device according to claim 1, wherein the matching fluid (7, 21, 53) is a composite vapor comprising at least two components (50, 51).

3. The device according to claim 1, wherein the vapor comprises a first scattering component (51) dissolved in droplets of a second component (50).

4. The device according to claim 2, wherein the matching fluid comprises a component with a transport mean-free path, lₜᵣₐ, below 3 millimeter.

5. The device according to claim 3, wherein the size of the droplets of the second component is larger than the transport mean-free path of the scattering component of the first component.

6. The device according to claim 3, wherein the ratio between the refractive index of the first scattering component and the second component is larger than 1.5.

7. The device according to claim 3, wherein the first scatting component is titanium dioxide and the second component is water.

8. The device according to claim 3, wherein a dye is added to the second component.

9. The device according to claim 1, further comprising a nebulizer (27) and wherein the vapor is generated by the nebulizer.

10. The device according to claim 1, further comprising a device (28) for generating sound waves for randomizing the position of the particles in the vapor.

11. The device according to claim 1, wherein the radiation source irradiates the turbid medium at a selected wavelength such that at the selected wavelength one or more selected optical properties of the matching fluid substantially matching the corresponding optical properties of the turbid medium.

12. The device according to claim 1, further comprising a processing unit for deriving an image of the turbid medium from the measured intensities.

13. The device according to claim 1, wherein the one or more of the optical properties are one or more attenuation coefficients.

14. The device according to claim 1, wherein the one or more of the optical properties are such that the scattering and absorption properties are higher than those of water.

15. The device according to claim 1, wherein the vapor is a mist or smoke.

16. A method of imagining a turbid medium, the method comprising:
arranging (60) in a holder the turbid medium and a matching fluid;
irradiating (61) the turbid medium and the matching fluid with one or more radiation sources;
measuring (62) the intensity of the radiation by one or more photodetectors;
wherein the matching fluid is selected as a vapor with one or more optical properties of the matching fluid substantially matching the corresponding one or more optical properties of the turbid medium.
